(19) United States
(12) Patent Application Publication
(10) Pub. No.: US 2010/0152764 A1
(43) Pub. Date: Jun. 17, 2010

(54) DEVICE FOR TREATING OBESITY

(76) Inventor: Dominique Merkle, Schondorf (DE)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 12/441,205

(22) PCT Filed: Jul. 27, 2007
(86) PCT No.: PCT/EP2007/006439

§ 371 Date: Dec. 3, 2009

(30) Foreign Application Priority Data
Sep. 13, 2006 (DE) 10 2006 042 955.9

(51) Int. Cl. A61M 29/00

(52) U.S. Cl. 606/192

(57) ABSTRACT

A device for treating obesity in humans is described, with a balloon arrangement that can be introduced into the human stomach and that can be filled with a fluid and emptied inside the stomach for controlled reduction of the stomach volume; with at least one fluid line, which is connected in a fluid tight manner to the balloon arrangement and via which the balloon arrangement can be filled and emptied transcutaneously; with at least one securing means provided along the fluid line in order to fix the fluid line to skin and tissue layers enclosing the stomach; and with at least one valve unit provided along the fluid line. The balloon arrangement has a first means that can be filled with the fluid and assumes a balloon-like configuration in a filled state, a second fillable means which, in a filled state, assumes a ring-shaped or loop-shaped configuration and radially encloses a clear internal diameter, and the first and second means are connected to each other in so that the first means extends into a first half space via a plane enclosed by the ring-shaped or loop-shaped configuration of the filled second means.

### BACKGROUND OF THE INVENTION

1. Field of the Invention

The invention relates to a device for treating obesity in humans, with a balloon-like arrangement that can be introduced into the human stomach and that can be filled with a fluid and emptied inside the stomach for controlled reduction of the stomach volume, with at least one fluid line, which is connected in a leaktight manner to the balloon arrangement and via which the balloon arrangement can be filled and emptied transcutaneously, with at least one securing means provided along the fluid line in order to fix the fluid line to skin and tissue layers enclosing the stomach, and with at least one valve unit provided along the fluid line.

2. Description of the Prior Art

Pathological obesity (adiposis) has been recognised by the WHO since 1997 as a chronic illness and is the fastest growing health risk in the world. The underlying cause of adiposis is a long-term positive energy balance as a result of mismanaged intake of food with a background of genetic factors. To estimate the extent of the adiposis, the Body Mass Index (BMI [kg/m²]) is calculated from the weight and body size of the patient (kg/m²). A BMI of greater than 29.9 is considered a symptom of adiposis, which is subdivided with increasing BMI into four different degrees of severity of the illness. Adiposis has for a long time reached epidemic levels, especially in the industrialized nations. In Germany, the prevalence of adiposis (BMI>29.9) has increased continually for many years. Approximately 50% of adult men are currently overweight with a BMI of >24.9 and approx. 18% are obese with a BMI>29.9. Among adult women, approximately 35% are overweight and almost 20% are obese. Particularly shocking is the rapid rise among children and youths in recent years.

In the USA, more than 4 million people suffer from grade III adiposis, that is BMI>40 and in Germany almost 1.2 million suffer from grade III adiposis. In addition to the extent of the excess weight, which is determined by means of the BMI, the fat distribution pattern defines the metabolic and cardiovascular health risks. The visceral fat mass correlates particularly closely with cardiovascular risk factors and complications. A simple measure for assessing the visceral fat deposit is measuring the waist circumference. An abdominal adiposis is present with a waist circumference of >87 cm in women and >101 cm in men. The waist circumference should always be measured in persons with a BMI>25 kg/m².

Excess weight and adiposis constitute a serious health risk, especially because of the comorbidities associated with adiposis. These include in particular: cardiovascular illnesses (cardiovascular disease, stroke, cardiac insufficiency), arterial hypertension, disruption of carbohydrate metabolism (type 2 diabetes mellitus, insulin resistance), fat metabolism disorders, hyperuricaemia/gout, obstructive sleep apnoea, reflux disease, degenerative changes in the supporting and connective tissue systems, increased incidence of carcinomas in women: cervix, ovaries, breast and kidney, increased incidence in carcinomas in men: prostate, colon, pancreas and kidney, and stress incontinence. Furthermore, excess weight and adiposis often lead to restrictions to the activities of everyday life, reduced quality of life, increased risk of accident, increased risk of complications during pregnancy, psychosocial consequences with tendencies to depression and anxiety, social discrimination, loss of self-esteem and social isolation.

Different therapy options are available to combat the symptoms of adiposis, which are briefly described below:

### Conservative Therapy

The basis of each weight management system should be a basic program which combines nutritional, movement and behavioral therapy. A distinction is to be made fundamentally between 2 phases, a 1st phase with the focus on weight reduction, and a 2nd phase in which the emphasis is placed on maintaining the reduced weight. Maintenance of weight is to be achieved permanently in all cases only with long-term change of nutrition to a varied, balanced diet as recommended by the German Nutrition Society. The lowest number of patients reach this goal, which explains the steady increase in the adiposis.

### Surgical Therapy

There are a multiplicity of different surgical measures for adiposis available, the use of which should be weighted. The degree of the adiposis and other factors (comorbidities) play an important role here, but the decision for a specific procedure should of course always be taken in an individual agreement between doctor and patient. The currently most customary methods are described below.

### “Gastric Balloon”

The endoscopic implantation of a gastric balloon is recommended as a rule for patients with grade I adiposis, that is, BMI between 30 and 34.9. The fluid-filled balloon remains in the stomach for 6 months and is used to initiate weight reduction. The reduction in weight to be expected in patients with grade I adiposis is approx. 10-20 kg within these 6 months. If the patient has not fundamentally changed his or her eating behavior during this time, what is known as the yo-yo effect with renewed increase in weight will quickly occur. According to experience, a sensible therapy for weight reduction and maintenance must be carried out over a plurality of years in order to be successful in the long term.

US 2005/0004430 A1 discloses an endoscopic instrument with which it is possible to place a gastric balloon in the stomach through the oesophagus, on the distal end of which an elastically inflatable balloon body is provided, which can be filled with a fluid upon being placed within the stomach, sealed off in a gas-tight manner with respect to the endoscope and finally separated from the latter. The balloon which is introduced into the stomach reduces the natural stomach volume and gives the patient a rapid feeling of satiation even with a small intake of food.

US 2005/0159769 A1 likewise discloses a balloon system for implantation in the stomach which, in contrast to the document cited above, can be filled with a medium via a supply line which runs transcutaneously. The filling level is controlled by a control unit, which is provided extracorporally and by means of which the balloon inside the stomach assumes a size which can be adjusted individually.

Although the above-described balloon which is introduced into the stomach remains connected to the transcutaneously running connection line, a defined placing of the balloon within the stomach is not guaranteed. This disadvantage is overcome in the gastric balloon according to U.S. Pat. No. 6,454,785 B2, which, although likewise connected via what is known as a filling catheter which projects transcutaneously through the abdominal wall, can additionally be fixed securely relative to the abdominal wall by means of anchoring valves. This means that the balloon is placed at a defined point inside the stomach, in which it is ensured that the balloon does not close off the oesophagus which opens into the stomach or the lower region of the pyloric orifice.

In addition to the above-described methods of using a gastric balloon, operative methods for treating obesity are also known, which are classified into purely restrictive and combinational methods with a more or less pronounced malabsorptive component.

With purely restrictive methods such as the use of a gastric band or gastroplasty, a small pouch is formed from the upper portion of the stomach directly below the cardia, which is able to empty via a narrow duct (stoma) into the rest of the stomach. This measure means that a feeling of satiation is produced even after a small intake of food. In combinational methods such as the stomach bypass and biliopancreatic diversion, a small partial stomach is connected to a small intestine loop of varying length. The length of small intestine which is functional decides the extent of the malabsorption. Weight loss therefore takes place on the one hand by means of the restriction of food intake (restrictive component), and on the other hand by means of the shortening of the functional small intestine (malabsorption component).

One criterion for deciding which operative method should be given preference is certainly the existing BMI before the operation and the safety of the operation to be carried out. Whereas the direct operative risk with gastric band implantation is very low, complication rates of 10-20% are indicated in the long term, which result in difficult follow-up operations. The perioperative risk of the stomach bypass operation and biliopancreatic diversion is up to 10% for major complications, however many fewer complications are seen in the long term. A decisive disadvantage of these operations is that they can only be reversed with difficulty.

Common to all the methods is a clear weight reduction between 60 and 80% of the excess weight within the first 2 years. This entails a considerable reduction in supporting and moving apparatus complaints, improvement in cardiovascular symptoms, and improvement, even disappearance in individual cases, of diabetes mellitus.

Whereas conservative adiposis programs are considered successful if 50% of participants have a weight reduction of more than 5% and 20% have a weight reduction of more than 10% within a year, a 60-80% reduction in excess weight is the rule following bariatric surgery. The disadvantages are however relatively high complication rates occurring operatively or in the long term.

### SUMMARY OF THE INVENTION

The invention is based on development of a device according to the above-cited U.S. Pat. No. 6,454,785 B2 for treating obesity in humans, with a balloon arrangement that can be introduced into the human stomach and that can be filled with a fluid and emptied inside the stomach for controlled reduction of the stomach volume in such a manner that a stimulation of the stomach for producing a feeling of satiation is dispensed more finely and brought about more quickly, without straining the stomach by excessive dilation of the gastric balloon in the process. The device should offer the patient in the most gentle possible manner the opportunity to learn healthy eating behavior without being restricted in everyday activities. In order to remove the device from the stomach, the device should not affect stomach-intestine continuity. Finally, the existing great demand for the device to be offered as cost-effectively as possible ensures that the measure can be used as extensively as possible.

The device according to the invention for treating obesity in humans includes a balloon arrangement with a first means which can be filled with the fluid and assumes a balloon-like configuration in a filled state, which in a preferred embodiment variant has an outer contour which is matched to the upper inner stomach wall region. Furthermore, a second means is provided which can be filled with the fluid and in a filled state assumes a ring-shaped or loop-shaped configuration and radially encloses a clear internal diameter. The first and second means are connected to each other in such a manner that the first means extends into a first half space via a plane enclosed by the ring-shaped or loop-shaped configuration of the filled second means.

The first and second means, which can be filled in each case via a fluid line with a gaseous or liquid fluid, in each case provide an elastic shell whose geometry determines the size, shape and arrangement of the two means within the stomach. For safety reasons, the elastic shells consist of a plurality, that is, two, three or more, individual elastic layers which each together enclose an inner lumen. Both means are preferably connected to each other in such a manner that they can be filled and emptied via a single fluid line. Of course, it is however also possible for the first and second means to be filled with corresponding fluid separately from each other with in each case one fluid line, in order for example to be able to adjust the filling level of the individual means individually and independently from each other.

In contrast to the previously known gastric balloon systems, the balloon arrangement according to the invention includes the combination of at least two means which can be filled with a fluid and have different tasks to perform according to their shape and positioning inside the stomach after being correspondingly introduced into the stomach. The second means, which in the filled state assumes a ring-shaped or loop-shaped configuration, is formed in shape and size in such a manner that the outer contour of the second means in the filled state bears with slight pressure against the inner wall of the stomach along a cross-sectional plane through the body of the stomach, so that the stomach volume is divided by the ring-shaped or loop-shaped second means into an upper and lower stomach region, with the oesophagus opening into the upper stomach region and the lower stomach region being connected at its exit with the duodenum. The upper and lower stomach regions are only connected fluidically by the clear internal diameter which is radially enclosed by the second means in the filled state, the clear opening width of which internal diameter can be varied by the filling level of the second means. In this manner it is possible to use the filling level of the second means to control the stomach throughput and even completely suppress it when the second ring-shaped or loop-shaped means is completely filled.

In a preferred embodiment, the second means is configured as a ring which can be inflated and deflated elastically, whose ring inner region has a greater elasticity than the remaining ring region, so that when the ring-shaped shell is filled, the ring region which faces the clear internal diameter is able to expand more than the remaining ring. This ultimately results in the clear internal diameter being reduced to a minimum when the ring-shaped second means is completely filled, as a result of which the throughput from the upper stomach region to the lower stomach region can even be completely suppressed. In addition to the function as a type of limiter for throughput through the stomach, the second means is used as a mechanical support for the first means, which has a balloon-like configuration in the filled state and is connected to the second means, which has a ring-shaped or loop-shaped configuration in the filled state, in such a manner that the first means is arranged completely in the upper stomach region without closing off the clear internal diameter of the second means. The balloon-like first means which, as already mentioned above, preferably has an outer contour which is matched to the upper inner stomach wall in the filled state, is preferably fastened to a radially outer ring or loop region of the second means and depending on the filling level nestles against the stomach inner wall of the fundus, the targeted stimulation of which triggers the feeling of satiation.

If for example it is necessary to bring about a feeling of satiation in a patient as quickly as possible, that is, with only a small quantity of food, the filling level of the ring-shaped or loop-shaped second means is selected to be maximal so that no or only a low throughput through the stomach exists. In this case the food remains for longer in the upper stomach region, as a result of which the balloon-like first means very quickly effects a stimulation at the upper stomach wall region and effects a feeling of satiation. Furthermore, the bolus is passed through the narrowed clear internal diameter of the second means to the lower region of the stomach, in order to be finally transported from the stomach to the small intestine.

In order to fasten the balloon arrangement which consists of the first and second means, the second means provides on a side which faces away radially from the clear internal diameter a holding element which clamps an area and is used primarily for bearing against an inner wall region of the stomach. The holding element braces the peripheral outer edge of the second means in a local region, in order to fasten it relative to a transcutaneous through-flow duct through the abdominal wall together with the first means which is attached to the second means or to the holding element itself. To this end, the at least one fluid line for filling at least the first and second means is fixed securely to the holding element which clamps the area and in the implanted state of the balloon arrangement transcutaneously penetrates the stomach wall and the abdominal wall which encloses the stomach. Instead of a single fluid line, it is likewise possible to connect two or more separate fluid lines, which are preferably combined to form a fluid line bundle, to the holding element which clamps the area. When the transcutaneously running fluid line is correspondingly positioned, the ring-shaped or loop-shaped second means is arranged approximately centrally with respect to the longitudinal extent of the stomach, from where the first means which has a balloon-like configuration on filling can extend into the upper stomach region. The holding element, which can have a plate-like, star-like, disc-like or cushion-like configuration is thus used together with the fluid line as a fixing means of the balloon arrangement in the stomach interior.

A fixing element is provided proximally at a distance of approximately 1.5 to 2 cm from the above-described holding element along the fluid line as a type of counter element, which is preferably configured as a type of inflatable body which can be filled with a fluid and emptied, and in the empty state has a diameter in the order of magnitude of the line diameter which is associated with the fluid line. If the inflatable body is filled, the inflatable body diameter widens and assumes a size which is greater than the line diameter of the fluid line. As can be seen in detail with reference to an exemplary embodiment below, the distance along the fluid line provided between the holding element and the inflatable body becomes smaller with increasing filling of the inflatable body so that in the implanted state the stomach wall and musculature of the abdominal wall can be clamped between the holding element and fixing element so that the fluid line remains fixed relative to the abdominal wall and, in connection with this, the balloon arrangement implanted inside the stomach ultimately remains fixed. The fluid element, which is configured as an inflatable body, is to be filled preferably by means of an additional fluid line, which, like the fluid line for filling and emptying the first and second means, is guided outwards extracorporally.

Alternatively, the inflatable body along the fluid line can be configured as a material section with increased elasticity compared to the rest of the fluid line, which material section assumes an enlarged shape compared to the rest of the fluid line both radially and axially with respect to the fluid line owing to pressurized filling of the fluid line. In this embodiment variant, no separate fluid line is required to fill the inflatable body.

At least one sealing element, for example in the form of a ball valve, which can seal off the fluid line in a fluid-tight manner and effects a gas-tight seal with respect to the fluid pressure which acts on the distal end, is preferably situated proximally along the fluid line with respect to the fixing element. A closing cap is provided at the proximal end of the fluid line, which closing cap projects extracorporally above the outer skin layer when the device is implanted. A microchip is preferably integrated inside the closing cap, which microchip can actuate the ball valve provided along the fluid line by means of a corresponding control unit in order to vary the filling level of the balloon arrangement provided intracorporally inside the stomach according to individual requirements. To this end, the fluid line is to be connected to a corresponding fluid reservoir when the balloon arrangement is filled correspondingly.

In a further preferred embodiment, the balloon arrangement which is to be introduced intracorporally inside the stomach provides in addition to the first and second means a third means which can be filled with fluid, which in the filled state assumes a balloon-like shape in the same manner as the first means and is connected to the first and second means in such a manner that the third means extends into a second half space which lies opposite the first half space, separated by the plane. When the balloon arrangement is implanted inside the stomach this results in the third means, which in the filled state has a balloon-like configuration, being arranged in the lower stomach region and can support both the second but in particular also the first means within the stomach. Depending on the embodiment variant, it is possible to fill and empty all three balloon-like means, which in each case preferably consist of a multi-layer elastic shell and enclose an inner lumen, with a single fluid line, with the lumens of all three means being fluidically connected to each other and communicating with each other. Alternatively, it is however possible to adjust the filling level of the individual means independently of each other by separate fluid lines. In order to achieve this variant, it is recommended that the fluid line be configured as a bundle in each case consisting of a plurality of separate fluid lines. To do this it is, however, necessary to provide corresponding valve units, preferably in the form of ball valves, along each individual fluid line.

To implant the device according to the invention, it is assumed below that the balloon arrangement has three means which are connected fluidically to each other and can be filled with a gaseous or liquid fluid via a fluid line. The fluid line is, as described above, preferably attached in a fluid-tight manner to the ring-shaped or loop-shaped means by means of a corresponding holding element and has along its proximal extent a fixing element which is configured as an inflatable body and at least one valve unit which is preferably configured as a ball valve. The device according to the invention is implanted in the stomach by means of percutaneous endoscopic gastrostomy (PEG) through the oesophagus, with an auxiliary catheter which is introduced into the stomach transcutaneously by means of transgastric puncture being used for the implantation along the oesophagus. Such an implantation method has been standard in medicine for a long time and is associated with only low complication rates of 0.3 to 1% and thus entails a considerably lower risk to the patient than all the other above-mentioned surgical methods. It is possible using the proximal end, which is brought out extracorporally transcutaneously by means of the auxiliary catheter, of the fluid line to align the triple-lumen balloon arrangement, which is introduced inside the stomach, and then fix it to the musculature of the abdominal wall. Using the holding element, for example in the form of a holding plate, which is attached to the ring-shaped or loop-shaped second means, it is possible to place the balloon arrangement inside the stomach in the above-described manner. The correct placement of the balloon arrangement within the stomach can be checked with the aid of diagnostic techniques which are known per se, such as x-ray or magnetic technologies. To this end, it is advantageous to provide at least the holding element with a detectable platelet of suitable material.

The individual inflatable lumens are then filled in stages, preferably under endoscopic control, until a desired filling level of all three lumens introduced into the stomach is achieved. In order to finally fix and position the balloon arrangement within the stomach, the fixing element which is attached proximally to the musculature of the abdominal wall and configured as an inflatable body is dilated in a controlled manner in order to ensure a secure seating of the balloon arrangement relative to the stomach wall and abdominal wall. As an alternative to the above-described embodiment variant of the inflatable body in which a lumen is arranged separately relative to the fluid line and can be filled separately, it is likewise possible to produce the fluid tube along its proximal extent at a distance of approx. 1.5 to 2 cm from the balloon arrangement from a material which has a higher elasticity than in the region of the rest of the fluid tube. If a valve unit is provided along the fluid line at the distal end from the highly elastic fluid tube region, which valve unit is closed correspondingly after a desired filling level is achieved in the lumens introduced inside the stomach, a further fluid filling along the fluid tube leads to a local radial and axial expansion of the fluid tube, as a result of which the musculature of the abdominal wall, which is situated between the holding element and the inflatable body which arises due to the filling, is enclosed locally on both sides.

To close, the fluid tube, which projects extracorporally above the skin surface, is fastened just above the skin surface and sealed off with a closing cap.

The device according to the invention enables an unhindered application without restriction in the movement behavior of the patient. It can be used particularly advantageously for long term treatments for learning healthy eating habits and moreover allows learned eating behavior to be checked by reducing the filling level of the individual balloon-like lumens in a controlled manner and allowing the stomach to assume the task of natural food utilization largely without artificial stimulation by the balloon arrangement. If it is established that the taught eating behavior has not been sufficiently put into practice, the individual lumens of the balloon arrangement can be suitably filled again.

If the treatment has led to a considerable reduction of the excess weight as desired and the patient is able to keep his or her weight constant over a long period with an empty balloon arrangement, it is furthermore possible to remove the balloon arrangement according to the solution extracorporally without any problems. To this end, the balloon arrangement is completely emptied, the fluid tube is cut endogastrically just above the holding plate or holding element and endoscopically extracted with a customary endoscopic loop. The remaining fluid tube is pulled out of the subcutaneous tissue by the closing cap. The above measures can be carried out in a minimally invasive manner, with the stomach-intestine continuity being unrestrictedly maintained.

The device according to the invention allows extensive application in all industrialized nations by using the PEG method which is known per se and standardized so that it can be expected that there is a high acceptance potential among persons involved in the treatment of adiposis owing to largely negligible risks.

### BRIEF DESCRIPTION OF THE DRAWINGS

The invention is described below by way of example without restriction of the general inventive idea using exemplary embodiments with reference to the drawings. In the figures,

FIG. 1 shows a schematized illustration of a balloon arrangement implanted in the stomach; and

FIG. 2 shows a cross-sectional diagram through the ring-shaped or loop-shaped second means which can be filled with a fluid.

### DETAILED DESCRIPTION OF THE INVENTION

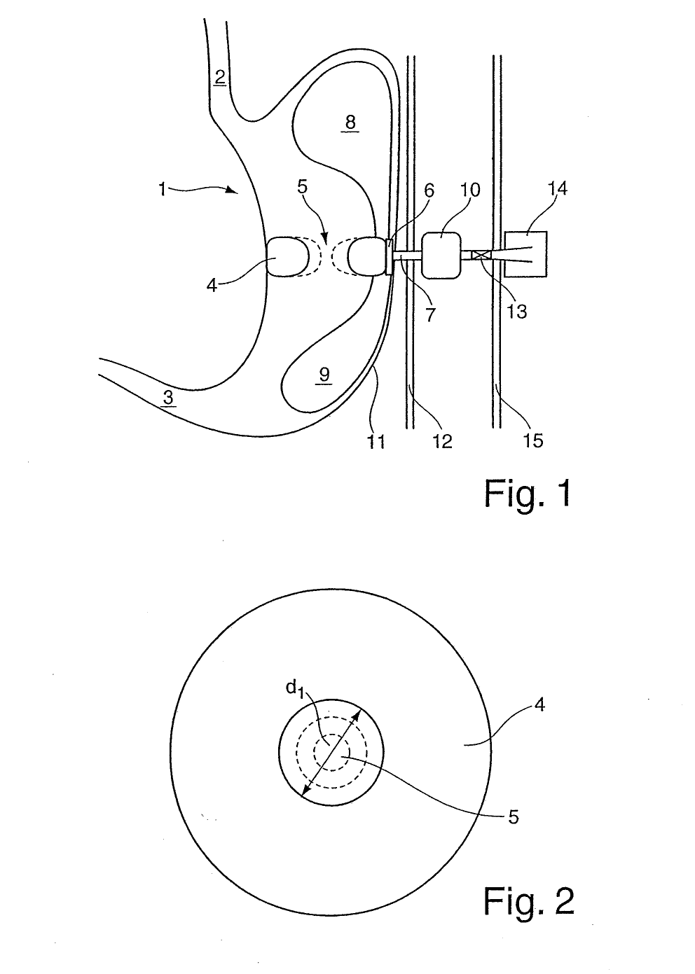

FIG. 1 shows a schematized longitudinal section through the stomach 1 of a human, in the upper region of which the oesophagus 2 opens and to the lower region of which the small intestine or duodenum 3 adjoins. A balloon arrangement is introduced inside the stomach 1, which balloon arrangement consists of the three following means which are in each case configured as balloon bodies. A second means 4 is provided centrally inside the stomach 1, which second means has a ring-shaped configuration in the filled state and radially encloses a clear internal diameter 5 as a function of its filling level. The means 4, which has a ring-shaped configuration in the filled state, adjoins the inner wall of the stomach 1 in a largely fluid-tight manner with its peripheral circumferential edge and thus divides the stomach into an upper and a lower stomach region. Both stomach regions are only connected to each other fluidically by the clear internal diameter 5 of the means 4 which can be filled, with the clear internal diameter 5 being reduced to a minimum when the means 4 is completely filled with a fluid which can be basically gaseous or liquid. In this case there is virtually no possibility of throughput from the upper to the lower stomach region. A holding element 6 is provided on the peripheral outer side of the means 4 which faces the abdominal wall 12 for the purpose of positioning and fixing the balloon arrangement within the stomach 1, which holding element is configured as a holding plate in the exemplary embodiment shown schematically in FIG. 1. The holding element 6 is used on the one hand as a bearing face or supporting face on the inner wall of the stomach 1, on the other hand as a securing means for the fluid line 7, via which the balloon arrangement can be filled with or emptied of a fluid.

Furthermore, what is known as a first means 8 is provided, which, as shown schematically in FIG. 1, has a balloon-like outer contour in the filled state, which is matched to the inner wall of the upper stomach region. The means 8 is attached in the peripheral region of the ring-like means 4 and when the means 4 is in the filled state projects in such a manner that the clear internal diameter 5 of the means 8 cannot be closed off. Since the feeling of satiation in humans is triggered by stimulation of the inner wall of the upper stomach region (fundus), the means 8 is used, depending on the filling level, for the fastest possible stimulation of the feeling of satiation which can be registered by the human. Alternatively, it is also possible to connect the first means 8 to the second means 4 by means of the holding element 6.

For improved support of the balloon arrangement inside the stomach 1, a third means 9 is also used, which can likewise be filled with the fluid via the fluid line 7 and in the filled state assumes a balloon-like shape which can nestle in a supporting manner against the inner wall of the lower stomach region. The third means 9 is connected to the ring-shaped means 4 at its peripheral circumferential region in the same manner as the first means 8 or, as mentioned above as an alternative configuration, fastened to the holding element 6.

Depending on the embodiment variant, it is possible to fill with fluid or empty all three above-described means 4, 8, 9 which can be filled with a fluid via a single fluid line 7. It is likewise conceivable to implement the fluid line 7 as a line bundle through which each individual means 4, 8, 9 can be filled with a fluid separately from each other so that different filling levels can be produced in the individual lumens of the means 4, 8, 9. As already indicated, the ring-shaped means 4 is used, in addition to mechanically supporting the first means 8 which is arranged in the upper stomach region, also as a throughput-limiting means owing to the possibility of varying the clear internal diameter 5. To this end, a cross section through the ring-shaped means 4 is shown in FIG. 2, which at a first filling level delimits a clear internal diameter 5, which has a diameter d1 and with which a maximum throughput from the upper to the lower stomach region is ensured. With an increasing filling level the clear internal diameter 5 narrows, as a result of which the throughput from the upper to the lower stomach region is greatly reduced. Depending on the embodiment of the ring-shaped means 4, it is possible to reduce the output diameter d1 to a minimum in order to trigger the feeling of satiation with very small quantities of food taken in. In order to realize the ring-shaped means 4, it is possible to provide a lumen which can be filled separately in the region of the ring-shaped means 4 which faces the clear internal diameter 5, which lumen is defined by an elastic shell which faces the clear internal diameter 5 and whose elasticity is greater than the elastic shell which encloses the ring-shaped lumen of the ring-shaped means 4. To this end, it is necessary to provide an additional fluid line for separately filling such a separate lumen which lies in the ring-shaped means 4.

Alternatively, it is possible to configure the ring-shaped means 4 in each case with different elastic regions. The region which faces the clear internal diameter has a greater elasticity than the remaining region of the ring-shaped means 4. In this case the ring-shaped means 4 can also expand preferably radially inwards with increasing filling and define individually the clear internal diameter 5.

For therapeutic reasons, it is furthermore advantageous to provide at least the surface region of the ring-shaped means 4 which faces the clear internal diameter 5 with a chemical or biological, activatable layer. For example a layer which has glucose receptors, which can be activated with intake of a certain liquid.

For the purpose of securely fastening the balloon arrangement, which is introduced in the interior of the stomach 1, a fixing element 10 is furthermore provided along the fluid line 7 (see FIG. 1) at a distance of approximately 1.5 to 2 cm proximally with respect to the holding element 6, which fixing element is preferably configured as an inflatable body and when filled with a fluid is subjected to an expansion which is oriented both radially and axially with respect to the fluid line 7 so that the stomach wall 11, which lies between the holding element 6 and the fixing element 10, and the musculature of the abdominal wall 12 are pressed against each other.

Furthermore, at least one valve unit 13 is provided along the fluid line 7, which valve unit is configured as a self-sealing valve, preferably in the form of a ball valve. The ball can be pressed into a valve seat in a fluid-tight manner by a pressure which is produced on the balloon arrangement side and can be transferred into an open valve position by axial displacement of the ball against the prevailing internal pressure on emptying. If separate fluid lines are provided correspondingly for filling the individual means 4, 8, 9, a corresponding valve is to be introduced along each individual fluid line. Finally, a closing cap 14 closes off the fluid line extracorporally, which is provided flush with the skin surface 15 when the balloon arrangement is implanted. The closing cap 14 has a connection flange for a fluid reservoir, from which fluid is provided for filling the balloon arrangement in a dosed and controlled manner. A microchip provided within the closing cap 14 can moreover initiate individually specifiable filling patterns in a program-controlled manner as a function of a specifiable rule. In this manner, the teaching of a new type of eating behavior can be considerably accelerated.

### LIST OF REFERENCE SYMBOLS

1 Stomach
2 Oesophagus
3 Small intestine
4 Second ring-shaped or loop-shaped means
5 Clear internal diameter
6 Holding element
7 Fluid line
8 First means
9 Third means
10 Fixing element
11 Stomach wall
12 Musculature of the abdominal wall
13 Valve unit
14 Closing cap
15 Skin surface

1-25. (canceled)

26. A device for treating obesity in humans, comprising:
a balloon arrangement for introduction into the human stomach and which is fillable with a fluid and emptied inside the stomach for a controlled reduction of the stomach volume;
at least one fluid line, which is connected in a fluid tight manner to the balloon arrangement, and via which the balloon arrangement is filled and emptied transcutaneously;
at least one securing means provided along the fluid line for fixing the fluid line to skin and tissue layers enclosing the stomach;
at least one valve provided along the fluid line, and wherein:
the balloon arrangement includes a first means which is fillable with the fluid and assumes a balloon-like configuration in a filled state and a second fillable means which, in a filled state, assumes a ring-shaped or loop-shaped configuration and radially encloses a clear internal diameter; and
the first and second means are connected to each other so that the first means extends into a first half space via a plane enclosed by the ring-shaped or loop-shaped configuration of the filled second means.

27. A device according to claim 26, comprising:
a third fillable means, which in a filled state, assumes a balloon-like shape and is connected to the first and second means so that the third means extends into a second half space which lies opposite to the first half space and is separated by the plane.

28. A device according to claim 26, wherein:
the first and second means enclose lumens, which communicate with each other fluidically, and are filled and emptied together through the at least one fluid line.

29. A device according to claim 27, wherein:
the first and second means enclose lumens, which communicate with each other fluidically, and are filled and emptied together through the at least one fluid line.

30. A device according to claim 28, wherein:
the first, second and third means enclose lumens, which communicate with each other fluidically, and are filled and emptied through the at least one fluid line.

31. A device according to claim 29, wherein:
the first, second and third means enclose lumens, which communicate with each other fluidically, and are filled and emptied through the at least one fluid line.

32. A device according to one of claim 26, wherein:
the first and/or second means has on a side, which faces radially away from a clear internal diameter, a holding element which clamps an area and is used for bearing against an inner wall region of the stomach.

33. A device according to one of claim 27, wherein:
the first and/or second means has on a side, which faces radially away from a clear internal diameter, a holding element which clamps an area and is used for bearing against an inner wall region of the stomach.

34. A device according to one of claim 28, wherein:
the first and/or second means has on a side, which faces radially away from a clear internal diameter, a holding element which clamps an area and is used for bearing against an inner wall region of the stomach.

35. A device according to one of claim 29, wherein:
the first and/or second means has on a side, which faces radially away from a clear internal diameter, a holding element which clamps an area and is used for bearing against an inner wall region of the stomach.

36. A device according to one of claim 30, wherein:
the first and/or second means has on a side, which faces radially away from a clear internal diameter, a holding element which clamps an area and is used for bearing against an inner wall region of the stomach.

37. A device according to one of claim 31, wherein:
the first and/or second means has on a side, which faces radially away from a clear internal diameter, a holding element which clamps an area and is used for bearing against an inner wall region of the stomach.

38. A device according to claim 32, wherein:
the holding element is plate-shaped, ring-shaped or star-shaped.

39. A device according to claim 33, wherein:
the holding element is plate-shaped, ring-shaped or star-shaped.

40. A device according to claim 37, wherein:
the holding element is plate-shaped, ring-shaped or star-shaped.

41. A device according to claim 32, wherein:
the at least one fluid line is connected to the first and/or second means in the region of the holding element.

42. A device according to claim 38, wherein:
the at least one fluid line is connected to the first and/or second means in the region of the holding element.

43. A device according to claim 26, wherein:
a fixing element disposed along the fluid line proximally at a distance from the holding element.

44. A device according to claim 38, wherein:
a fixing element is disposed along the fluid line proximally at a distance from the holding element.

45. A device according to claim 41, wherein:
a fixing element is disposed along the fluid line proximally at a distance from the holding element.

46. A device according to claim 43, wherein
the fixing element comprises an inflatable body which is fillable with a fluid and emptied and when in an emptied state has a diameter a same size as a line diameter associated with the fluid line and when in the filled state has a diameter which is greater than the line diameter.

47. A device according to claim 46, wherein:
upon increasing filling the inflatable body reduces a distance between the holding element and the inflatable body.

48. A device according to claim 43, wherein:
the inflatable body encloses a lumen which is separate from the fluid line and is connected to a separate fluid line for filling and emptying.

49. A device according to claim 46, wherein:
the inflatable body encloses a lumen which is separate from the fluid line and is connected to a separate fluid line for filling and emptying.

50. A device according to claim 43, wherein:
the inflatable body along the fluid line includes a material section with increased elasticity compared to a remainder of the fluid line, and the material section assumes an enlarged shape, compared radially and axially to the remainder of the fluid line with respect to the fluid line upon pressurized filling of the fluid line.

51. A device according to claim 43, wherein:
at least one valve unit is disposed along the fluid line proximally with respect to the fixing element.

52. A device according to claim 51, wherein:
the at least one valve unit includes a self-sealing ball valve, including ball which can be pressed into a valve seat in a fluid-tight manner by a pressure prevailing on a balloon arrangement side and the valve can be transferred into an open valve position by axial displacement of the ball against the prevailing pressure for emptying the balloon arrangement.

53. A device according to claim 26, wherein:
the second means has a region which faces a clear internal diameter at least one lumen, which is separated from a remainder of the second means, so that the lumen provides on filling to a controlled narrowing of the clear internal diameter.

54. A device according to claim 53, wherein:
the at least one lumen is fillable with a fluid from a separate fluid line and is fillable independently of a filling level of a remainder of the second means.

55. A device according to claim 53, wherein:
the second means faces in a region a clear internal diameter providing an elastic shell for enclosing the at least one lumen of the second means, the elasticity of the elastic shell being greater than in a remaining region of the shell so that when the second means is filled with a fluid, the shell is expandable radially inwardly facing the clear internal diameter and provides a controlled narrowing of the clear internal diameter.

56. A device according to claim 53, wherein:
the first and second means have at least one elastic shell, which on filling defines the at least one lumen.

57. A device according to claim 26, wherein:
the first means has a shape matching an inner contour of a fundus so that at least a part of the fundus is touched after the balloon arrangement is implanted in the stomach; and
the second means has a radial outer contour which in the filled state is flush against an inner stomach wall of the stomach body.

58. A device according to claim 26, wherein:
a metallic or magnetic platelet, which is located by an x-ray or magnetic diagnostic technique, is provided on the first and/or second means.

59. A device according to claim 32, wherein:
the second means has a chemically or biologically active surface layer at least on a surface facing the clear internal diameter.

60. A device according to claim 59, wherein:
the active surface layer includes glucose receptors.

61. A device according to claim 28, wherein:
the fluid line has a closing cap which can be applied extracorporally on a proximal side of the fluid line.

62. A device according to claim 61, comprising a microchip integrated into the closing cap, which varies a filling level of at least the second means according to a specifiable rule.

63. A device according to claim 61, wherein:
fluid reservoir is connectable to the closing cap.
